(19) United States
(12) Patent Application Publication
(10) Pub. No.: US 2007/0037151 A1
(43) Pub. Date: Feb. 15, 2007

(54) Cd4+ human papillomavirus (hpv) epitopes

(76) Inventors: Lilia M. Babe, Emerald Hills, CA (US); Lawrence M. De Young, Montara, CA (US); Fiona A. Harding, Santa Clara, CA (US); Manley T. F. Huang, Palo Alto, CA (US); Scott D. Power, Palo Alto, CA (US); Marcia Stickler, San Jose, CA (US)

Correspondence Address: GENENCOR INTERNATIONAL, INC.;ATTENTION: LEGAL DEPARTMENT, 925 PAGE MILL ROAD, PALO ALTO, CA, 94304, US

(21) Appl. No.: 10/546,497

(22) PCT Filed: Apr. 23, 2004
(86) PCT No.: PCT/US04/12652

§ 371 Date: Oct. 10, 2006

Related U.S. Application Data
(60) Provisional application No. 60/466,235, filed on Apr. 28, 2003.

(51) Int. Cl. C12Q 1/70; C12Q 1/68

(52) U.S. Cl. 435/6; 435/5

(57) ABSTRACT

The present invention provides CD4+ T-cell epitopes in E6, E7 and E2 proteins from various strains of human papillomavirus (HPV). In some preferred embodiments, the present invention provides means for the development of HPV vaccines, in particular multivalent vaccines for the prevention of infection with high-risk HPV strains. In additional embodiments, the present invention provides means for the development of therapeutic vaccines against high-risk HPV types that prevent the development of benign and/or malignant tumors in infected individuals. The present invention further provides epitopes suitable for use in prophylactic and therapeutic vaccines.

### FIELD OF THE INVENTION

The present invention provides CD4+ T-cell epitopes in E6, E7 and E2 proteins from various strains of human papillomavirus (HPV). In some preferred embodiments, the present invention provides means for the development of HPV vaccines, in particular multivalent vaccines for the prevention of infection with high-risk HPV strains. In additional embodiments, the present invention provides means for the development of therapeutic vaccines against high-risk HPV types suitable for use in the prevention of the development of benign and/or malignant tumors in infected individuals. The present invention further provides epitopes suitable for use in prophylactic and/or therapeutic vaccines. In particularly m preferred embodiments, the present invention provides modified epitopes suitable for use in is prophylactic and/or therapeutic vaccines.

### BACKGROUND OF THE INVENTION

There is a large number of species-specific papillomaviruses within the genus Papillomavirus, including the human papillomaviruses (HPVs). Although the association between cutaneous HPV types and non-melanoma skin cancers has recently become of interest, most of the HPV research has been focused on the more than 30 sexually-transmitted HPVs (designated as “genital” HPV types). HPVs infect various tissues, including stratified squamous, metaplastic squamous, and columnar epithelia. There are two major groups of HPVs which tend to show some tissue tropism, with those in the “cutaneous” group infecting keratinizing epithelium and those in the “mucosal” group (e.g., genital types) infecting non-keratinizing epithelium. Within these groups, there are numerous types and strains. Although the majority of HPV infections are self-limited, it is clear that in a subset of genital HPV infections, malignant tumors develop.

Indeed, the association of HPV infection with cervical cancer has been firmly established. HPV infection is a necessary factor in cervical cancer, as HPV DNA is present in all cervical tumors (Wallboomers et al., J. Pathol., 189:1-3 [1999]; Munoz, J. Clin. Virol., 19:1-5 [2000]; and Bosch and de Sanjose, Curr. Oncol. Rep., 4:175-183 [2002]). Significantly, the prevalence of high-risk HPV strains (i.e., HPV strains that are known to be associated with a high risk for the development of malignancy) is very high in the general population. It is estimated that 30 to 60% of sexually active adult men and women are infected with at least one strain of genital HPV (Tindle, Nature Rev., 2:59-65 [2002]). One study reported that 13% of the United States adult population is seropositive for the HPV-16 IgG (Stone et al., J. Infect. Dis., 186:1396-1402 [2002]). This is important, as HPV-16 is associated with half of all cervical cancer cases worldwide. It is estimated that 0.1-1% of infections progress to neoplastic disease if left untreated (Tindle, supra). This is especially a problem in developing countries where 80% of cases occur due to lack of availability or cost of screening programs. Thus, there remains a great need for cost-effective screening and prevention programs suited toward the prevention of HPV infection and malignancy.

### SUMMARY OF THE MENTION

The present invention provides CD4+ T-cell epitopes in E6, E7 and E2 proteins from various strains of human papillomavirus (HPV). In some preferred embodiments, the present invention provides means for the development of HPV vaccines, in particular multivalent vaccines for the prevention of infection with high-risk HPV strains. In additional embodiments, the present invention provides means for the development of therapeutic vaccines against high-risk HPV types suitable for use in the prevention of the development of benign and/or malignant tumors in infected individuals. The present invention further provides epitopes suitable for use in prophylactic and/or therapeutic vaccines. In some preferred embodiments, the present invention provides the epitopes set forth in SEQ ID NOS:1-109. In particularly preferred embodiments, the present invention provides modified epitopes suitable for use in prophylactic and/or therapeutic vaccines.

The present invention further provides compositions and methods for the development of vaccine compositions directed against the E6 and E7 proteins of four high risk HPV strains (i.e., strains 16, 18, 45 and 56) and four moderate-risk HPV strains (i.e., strains 31, 33, 52 and 58). In additional embodiments, present invention further provides compositions and methods for the development of vaccine compositions directed against the E2 proteins of HPV strains 16, 18, 31 and 45. In some particularly preferred embodiments, the vaccine compositions are comprised of at least one epitope selected from the group selected from SEQ. ID NOS:1 through 109. In some alternatively preferred embodiments, the vaccine compositions comprise epitopes selected from at least one of the high-risk HPV strains and/or at least one of the moderate-risk HPV strains. Indeed, it is contemplated that the HPV vaccines of the present invention will find use in the treatment and prophylaxis of numerous HPV strains. It is not intended that the present invention be limited to any particular epitopes and/or vaccine compositions comprising any particular epitopes. Thus, in the various vaccine embodiments of the present invention, any combination of epitopes suitable for the intended use find use in the present invention.

The present invention further provides methods and compositions for the identification of epitopes in viruses such as HPV. In particular, the present invention provides applications for a T-cell assay system (the I-MUNE® assay) for the identification of CD4 T-cell epitopes in various HPV strains. In particularly preferred embodiments, the present invention provides CD4 T-cell epitopes of HPV strains 16, 18, 31, 33, 45, 52, 56, 58, including E6, E7 and E2 epitopes. However, it is not intended that the present invention be limited to these specific HPV strains nor these specific CD4 epitopes.

The present invention provides methods for the identification of HPV epitopes in the sequences of various HPV types, as well as the production of peptides which when incorporated into a HPV sequence, are capable of initiating the CD4⁺ T-cell response.

In some embodiments, the present invention provides methods for the identification of CD4⁺ T-cell epitopes in HPV sequences and the production of peptides that are capable of initiating the CD4⁺ T-cell response. In particular, the present invention provides means and compositions suitable for increasing the immunogenicity of HPV epitopes for use in HPV vaccine preparations.

In these embodiments, the present invention provides means for determining the T-cell responses of humans against various epitopes comprising a protein of interest. In additional embodiments, once the significant epitopes are identified using the I-MUNE® assay system described herein, the significant epitopes are altered to produce epitopes that induce an enhanced immune response to the protein.

Thus, as indicated above, the proteins of the present invention exhibit modified immunogenic responses (e.g., antigenicity and/or immunogenicity) when compared to the native proteins encoded by their precursor DNAs. For example, HPVs that exhibit increased immunogenic responses (e.g., variant HPV epitopes) find use in therapeutic and prophylactic vaccine compositions.

### DESCRIPTION OF THE FIGURES

FIG. 1 provides a graph showing the data obtained in the I-MUNE® assay with the peptide set from HPV E6.16.

FIG. 2 provides a graph showing the data obtained in the I-MUNE® assay with the peptide set from HPV E7.16.

FIG. 3 provides a graph showing the data obtained in the I-MUNE® assay with the peptide set from HPV E6.18.

FIG. 4 provides a graph showing the data obtained in the I-MUNE® assay with the peptide set from HPV E7.18.

FIG. 5 provides a graph showing the data obtained in the I-MUNE® assay with the peptide set from HPV E6.31.

FIG. 6 provides a graph showing the data obtained in the I-MUNE® assay with the peptide set from HPV E7.31.

FIG. 7 provides a graph showing the data obtained in the l-MUNE® assay with the peptide set from HPV E6.33.

FIG. 8 provides a graph showing the data obtained in the I-MUNE® assay with the peptide set from HPV E7.33.

FIG. 9 provides a graph showing the data obtained in the I-MUNE® assay with the peptide set from HPV E6.45.

FIG. 10 provides a graph showing the data obtained in the I-MUNE® assay with the peptide set from HPV E7.45.

FIG. 11 provides a graph showing the data obtained in the I-MUNE® assay with the peptide set from HPV E6.52.

FIG. 12 provides a graph showing the data obtained in the I-MUNE® assay with the peptide set from HPV E7.52.

FIG. 13 provides a graph showing the data obtained in the I-MUNE® assay with the peptide set from HPV E6.56.

FIG. 14 provides a graph showing the data obtained in the I-MUNE® assay with the peptide set from HPV E7.56.

FIG. 15 provides a graph showing the data obtained in the l-MUNE® assay with the peptide set from HPV E6.58.

FIG. 16 provides a graph showing the data obtained in the I-MUNE® assay with the peptide set from HPV E7.58.

FIG. 17 provides a graph showing the data obtained in the I-MUNE® assay with the peptide set from HPV E2.16.

FIG. 18 provides a graph showing the data obtained in the I-MUNE® assay with the peptide set from HPV E2.18.

FIG. 19 provides a graph showing the data obtained in the I-MUNE® assay with the peptide set from HPV E2.31.

FIG. 20 provides a graph showing the data obtained in the I-MUNE® assay with the peptide set from HPV E2.45.

### DESCRIPTION OF THE INVENTION

The present invention provides CD4+ T-cell epitopes in E6, E7 and E2 proteins from various strains of human papillomavirus (HPV). In some preferred embodiments, the present invention provides means for the development of HPV vaccines, in particular multivalent vaccines for the prevention of infection with high-risk HPV strains. In additional embodiments, the present invention provides means for the development of therapeutic vaccines against high-risk HPV types suitable for use in the prevention of the development of benign and/or malignant tumors in infected individuals. The present invention further provides epitopes suitable for use in prophylactic and/or therapeutic vaccines. In particularly preferred embodiments, the present invention provides modified epitopes suitable for use in prophylactic and/or therapeutic vaccines.

Indeed, development of such vaccines is contemplated to find use in obviating the need to develop population-based screening programs as well as reduce the incidence and prevalence of HPV infection. It is further contemplated that the development of therapeutic vaccines will find use in the prevention of tumor development. It is contemplated that the use of the present invention in prophylactic and/or therapeutic vaccines will reduce the morbidity and mortality associated with cervical and other cancers.

### DEFINITIONS

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Marham, The Harper Collins Dictionary of Biology, Harper Perennial, NY (1991) provide those of skill in the art with a general dictionaries of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also as used herein, the singular “a”, “an” and “the” includes the plural reference unless the context clearly indicates otherwise. To facilitate understanding of the invention, a number of terms are defined below.

As used herein, “HPV” and “human papillomavirus” refer to the members of the genus Papillomavirus that are capable of infecting humans. There are two major groups of HPVs (i.e., genital and cutaneous groups), each of which contains multiple virus “types” or “strains” (e.g. HPV 16, HPV 18, HPV 31, HPV 32, etc.). Of particular interest in the present invention are the HPV types that are associated with genital infection and malignancy.

As used herein, “prophylactic” and “preventive” vaccines are vaccines that are designed and administered to prevent infection, disease, and/or any related sequela(e) caused by or associated with a pathogenic organism, particularly HPV.

As used herein, “therapeutic” vaccines are vaccines that are designed and administered to patients already infected with a pathogenic organism such as at least one HPV strain. Therapeutic vaccines (e.g., therapeutic HPV vaccines) are used to prevent and/or treat the development of benign or malignant tumors in these infected individuals.

“Antigen presenting cell” as used herein refers to cells of the immune system which present antigen on their surface that is recognizable by T-cells. Examples of antigen presenting cells are dendritic cells, interdigitating cells, activated B-cells and macrophages.

The term “lymphoid” when used in reference to a cell line or a cell, means that the cell line or cell is derived from the lymphoid lineage and includes cells of both the B and the T lymphocyte lineages.

As used herein, the terms “T lymphocyte” and “T-cell,” encompass any cell within the T lymphocyte lineage from T-cell precursors (including Thy1 positive cells which have not rearranged the T-cell receptor genes) to mature T-cells (i.e., single positive for either CD4 or CD8, surface TCR positive cells).

As used herein, the terms “B lymphocyte” and “B-cell” encompasses any cell within the B-cell lineage from B-cell precursors, such as pre-B-cells (B220⁺ cells which have begun to rearrange Ig heavy chain genes), to mature B-cells and plasma cells.

As used herein, “CD4⁺ T-cell” and “CD4 T-cell” refer to helper T-cells, while “CD8⁺ T-cell” and CD8 T-cell” refer to cytotoxic T-cells.

As used herein, “B-cell proliferation,” refers to the number of B-cells produced during the incubation of B-cells with the antigen presenting cells, with or without antigen.

As used herein, “baseline B-cell proliferation,” as used herein, refers to the degree of B-cell proliferation that is normally seen in an individual in response to exposure to antigen presenting cells in the absence of peptide or protein antigen. For the purposes herein, the baseline B-cell proliferation level is determined on a per sample basis for each individual as the proliferation of B-cells in the absence of antigen.

With regard to a particular amino acid sequence, an “epitope” is a set of amino acid residues which is involved in recognition by a particular immunoglobulin, or in the context of T-cells, those residues necessary for recognition by T-cell receptor proteins and/or Major Histocompatibility Complex (MHC) receptors. In an immune system setting, in vivo or in vitro, an epitope is the collective features of a molecule, such as primary, secondary and is tertiary peptide structure, and charge, that together form a site recognized by an immunoglobulin, T-cell receptor or HLA molecule. Throughout this disclosure, “epitope” and “peptide” are often used interchangeably.

As used herein, “B-cell epitope,” refers to a feature of a peptide or protein that is recognized by a B-cell receptor in the immunogenic response to the peptide comprising that antigen (i.e., the immunogen).

As used herein, “altered B-cell epitope,” refers to an epitope amino acid sequence which differs from the precursor peptide or peptide of interest, such that the variant peptide of interest produces different (i.e., altered) immunogenic responses in a human or another animal. It is contemplated that an altered immunogenic response includes altered immunogenicity and/or allergenicity (i.e., an either increased or decreased overall immunogenic response). In some embodiments, the altered B-cell epitope comprises substitution and/or deletion of an amino acid selected from those residues within the identified epitope. In alternative embodiments, the altered B-cell epitope comprises an addition of one or more residues within the epitope.

As used herein “T-cell epitope” means a feature of a peptide or protein that is recognized by a T-cell receptor in the initiation of an immunologic response to the peptide comprising that antigen. Recognition of a T-cell epitope by a T-cell is generally believed to be via a mechanism wherein T-cells recognize peptide fragments of antigens which are bound to class I or class II Major Histocompatibility Complex (MHC) molecules expressed on antigen-presenting cells (See e.g., Moeller (ed.), Immunol. Rev., 98:187 [1987]). In some embodiments of the present invention, the epitopes or epitopic fragments identified as described herein find use in the detection of antigen presenting cells having MHC molecules capable of binding and displaying the epitopes or fragments. In some embodiments, the epitopes/epitopic fragments further comprise a detectable label (i.e., a marker) that facilitates the identification of cells that bind and/or display the epitope/epitopic fragment of interest.

As used herein, “T-cell proliferation,” refers to the number of T-cells produced during the incubation of T-cells with the antigen presenting cells, with or without antigen.

“Baseline T-cell proliferation,” as used herein, refers to the degree of T-cell proliferation that is normally seen in an individual in response to exposure to antigen presenting cells in the absence of peptide or protein antigen. For the purposes herein, the baseline T-cell proliferation level is determined on a per sample basis for each individual as the proliferation of T-cells in response to antigen presenting cells in the absence of antigen.

As used herein “altered immunogenic response,” refers to an increased or reduced immunogenic response. Proteins and peptides exhibit an “increased immunogenic response” when the T-cell and/or B-cell response they evoke is greater than that evoked by a parental (e.g., precursor) protein or peptide (e.g., the protein of interest). The net result of this higher response is an increased antibody response directed against the variant protein or peptide. Proteins and peptides exhibit a “reduced immunogenic response” when the T-cell and/or B-cell response they evoke is less than that evoked by a parental (e.g., precursor) protein or peptide. In some embodiments, the net result of this lower response is a reduced antibody response directed against the variant protein or peptide. In some preferred embodiments, the parental protein is a wild-type protein or peptide.

As used herein, the term “major epitope” refers to an epitope (i.e., a T-cell and/or B-cell epitope), wherein the response rate within the tested donor pool is at least three standard deviations above the mean background response rate.

As used herein, the term “moderate epitope” refers to an epitope (i.e., a T-cell and/or B-cell epitope), wherein the response rate within the tested donor pool is at least two standard deviations above the mean or three times the background.

As used herein, the term “minor epitope” refers to an epitope (i.e., a T-cell and/or B-cell epitope), wherein the response rate within the tested donor pool is at least twice the background.

As used herein, the term “significant epitope” refers to an epitope (i.e., a T-cell and/or B-cell epitope), wherein the response rate within the tested donor pool is equal to or greater than about three times the background response rate.

As used herein, a “weakly significant epitope” refers to an epitope (i.e., a T-cell and/or B-cell epitope), wherein the response rate within the tested donor pool is greater than the background response rate, but less than about three times the background rate.

As used herein, “background level” and “background response” refer to the average percent of responders to any given peptide in the dataset for any tested protein. This value is determined by averaging the percent responders for all peptides in the set, as compiled for all the tested donors. As an example, a 3% background response would indicate that on average there would be three positive (SI greater than 2.95) responses for any peptide in a dataset when tested on 100 donors.

The term “sample” as used herein is used in its broadest sense. However, in preferred embodiments, the term is used in reference to a sample (e.g., an aliquot) that comprises a peptide (i.e., a peptide within a pepset, that comprises a sequence of a protein of interest) that is being analyzed, identified, modified, and/or compared with other peptides. Thus, in most cases, this term is used in reference to material that includes a protein or peptide that is of interest.

As used herein, “protein of interest,” refers to a protein which is being analyzed, identified and/or modified. Naturally-occurring, as well as recombinant proteins, synthetically produced, variant and derivative proteins, all find use in the present invention.

As used herein, “protein” refers to any composition comprised of amino acids and recognized as a protein by those of skill in the art. The terms “protein,” “peptide” and polypeptide are used interchangeably herein. Amino acids may be referred to by their complete names (e.g., alanine) or by the accepted one letter (e.g., A), or three letter (e.g., ala) abbreviations. Wherein a peptide is a portion of a protein, those skill in the art understand the use of the term in context. The term “protein” encompasses mature forms of proteins, as well as the pro- and prepro-forms of related proteins. Prepro forms of proteins comprise the mature form of the protein having a prosequence operably linked to the amino terminus of the protein, and a “pre-” or “signal” sequence operably linked to the amino terminus of the prosequence.

As used herein, functionally similar proteins are considered to be “related proteins.” In some embodiments, these proteins are derived from a different genus and/or species, including differences between classes of organisms (e.g., a bacterial protein and a fungal protein). In additional embodiments, related proteins are provided from the same species. Indeed, it is not intended that the present invention be limited to related proteins from any particular source(s).

As used herein, the term “derivative” refers to a protein which is derived from a precursor protein by addition of one or more amino acids to either or both the C- and N-terminal end(s), substitution of one or more amino acids at one or a number of different sites in the amino acid sequence, and/or deletion of one or more amino acids at either or both ends of the protein or at one or more sites in the amino acid sequence, and/or insertion of one or more amino acids at one or more sites in the amino acid sequence. The preparation of a protein derivative is preferably achieved by modifying a DNA sequence which encodes for the native protein, transformation of that DNA sequence into a suitable host, and expression of the modified DNA sequence to form the derivative protein.

One type of related (and derivative) proteins are “variant proteins.” In preferred embodiments, variant proteins differ from a parent protein and one another by a small number of amino acid residues. The number of differing amino acid residues may be one or more, preferably 1, 2, 3, 4, 5, 10, 15, 20, 30, 40, 50, or more amino acid residues. In one preferred embodiment, the number of different amino acids between variants is between 1 and 10. In particularly preferred embodiments, related proteins and particularly variant proteins comprise at least 50%, 60%, 65%. 70%, 75%, 80%, 85%, 90%, 95%, 97%, 98%, or 99% amino acid sequence identity. Additionally, a related protein or a variant protein as used herein, refers to a protein that differs from another related protein or a parent protein in the number of prominent regions. For example, in some embodiments, variant proteins have 1, 2, 3, 4, 5, or 10 corresponding prominent regions that differ from the parent protein.

In one embodiment, the prominent corresponding region of a variant produces only a background level of immunogenic response. Some of the residues identified for substitution, insertion or deletion are conserved residues whereas others are not. In the case of residues which are not conserved, the replacement of one or more amino acids is limited to substitutions which produce a variant which has an amino acid sequence that does not correspond to one found in nature. In the case of conserved residues, such replacements should not result in a naturally-occurring sequence.

In some embodiments, the following cassette mutagenesis method finds use in the construction of the protein variants of the present invention, although other methods may be used. First, the naturally-occurring gene encoding the protein is obtained and sequenced in whole or in part. Then the sequence is scanned for a point at which it is desired to make a mutation (deletion, insertion or substitution) of one or more amino acids in the encoded protein. The sequences flanking this point are evaluated for the presence of restriction sites for replacing a short segment of the gene with an oligonucleotide pool which when expressed will encode various mutants. Such restriction sites are preferably unique sites within the protein gene so as to facilitate the replacement of the gene segment. However, any convenient restriction site which is not overly redundant in the protein gene may be used, provided the gene fragments generated by restriction digestion can be reassembled in proper sequence. If restriction sites are not present at locations within a convenient distance from the selected point (from 10 to 15 nucleotides), such sites are generated by substituting nucleotides in the gene in such a fashion that neither the reading frame nor the amino acids encoded are changed in the final construction. Mutation of the gene in order to change its sequence to conform to the desired sequence is accomplished by M13 primer extension in accord with generally known methods. The task of locating suitable flanking regions and evaluating the needed changes to arrive at two convenient restriction site sequences is made routine by the redundancy of the genetic code, a restriction enzyme map of the gene and the large number of different restriction enzymes. Note that if a convenient flanking restriction site is available, the above method need be used only in connection with the flanking region which does not contain a site.

Once the naturally-occurring DNA or synthetic DNA is cloned, the restriction sites flanking the positions to be mutated are digested with the cognate restriction enzymes and a plurality of end termini-complementary oligonucleotide cassettes are ligated into the gene. The mutagenesis is simplified by this method because all of the oligonucleotides can be synthesized so as to have the same restriction sites, and no synthetic linkers are necessary to create the restriction sites.

As used herein, “corresponding to,” refers to a residue at the enumerated position in a protein or peptide, or a residue that is analogous, homologous, or equivalent to an enumerated residue in a protein or peptide.

As used herein, “corresponding region” generally refers to an analogous position along related proteins or a parent protein.

As used herein, the term “analogous sequence” refers to a sequence within a protein that provides similar function, tertiary structure, and/or conserved residues as the protein of interest (i.e., typically the original protein of interest). In particularly preferred embodiments, the analogous sequence involves sequence(s) at or near an epitope. For example, in epitope regions that contain an alpha helix or a beta sheet structure, the replacement amino acids in the analogous sequence preferably maintain the same specific structure. The term also refers to nucleotide sequences, as well as amino acid sequences. In some embodiments, analogous sequences are developed such that the replacement amino acids show a similar function, the tertiary structure and/or conserved residues to the amino acids in the protein of interest at or near the epitope. Thus, where the epitope region contains, for example, an alpha-helix or a beta-sheet structure, the replacement amino acids preferably maintain that specific structure.

As used herein, “homologous protein” refers to a protein that has similar action, structure, antigenic, and/or immunogenic response as the protein of interest. It is not intended that a homolog and a protein of interest be necessarily related evolutionarily. Thus, it is intended that the term encompass the same functional protein obtained from different species. In some preferred embodiments, it is desirable to identify a homolog that has a tertiary and/or primary structure similar to the protein of interest, as replacement for the epitope in the protein of interest with an analogous segment from the homolog will reduce the disruptiveness of the change. Thus, in most cases, closely homologous proteins provide the most desirable sources of epitope substitutions. Alternatively, it is advantageous to look to human analogs for a given protein. For example, in some embodiments, substituting a specific epitope in one human HPV type with a sequence from another HPV or other species' papillomavirus results in the production of an HPV type that increases immunogenicity to a level suitable for use in vaccine preparations.

As used herein, “homologous genes” refers to at least a pair of genes from different, but usually related species, which correspond to each other and which are identical or very similar to each other. The term encompasses genes that are separated by speciation (i.e., the development of new species) (e.g., orthologous genes), as well as genes that have been separated by genetic duplication (e.g., paralogous genes). These genes encode “homologous proteins.”

As used herein, “ortholog” and “orthologous genes” refer to genes in different species that have evolved from a common ancestral gene (i.e., a homologous gene) by speciation. Typically, orthologs retain the same function in during the course of evolution. Identification of orthologs finds use in the reliable prediction of gene function in newly sequenced genomes.

As used herein, “paralog” and “paralogous genes” refer to genes that are related by duplication within a genome. While orthologs retain the same function through the course of evolution, paralogs evolve new functions, even though some functions are often related to the original one. Examples of paralogous genes include, but are not limited to genes encoding trypsin, chymotrypsin, elastase, and thrombin, which are all serine proteinases and occur together within the same species.

As used herein, “wild-type” and “native” proteins are those found in nature. The terms “wild-type sequence,” and “wild-type gene” are used interchangeably herein, to refer to a sequence that is native or naturally occurring in a host cell. In some embodiments, the wild-type sequence refers to a sequence of interest that is the starting point of a protein engineering project. The genes encoding the naturally-occurring (i.e., precursor) protein may be obtained in accord with the general methods known to those skilled in the art. The methods generally comprise synthesizing labeled probes having putative sequences encoding regions of the protein of interest, preparing genomic libraries from organisms expressing the protein, and screening the libraries for the gene of interest by hybridization to the probes. Positively hybridizing clones are then mapped and sequenced.

The term “recombinant DNA molecule” as used herein refers to a DNA molecule that is comprised of segments of DNA joined together by means of molecular biological techniques.

The degree of homology between sequences may be determined using any suitable method known in the art (See e.g., Smith and Waterman, Adv. Appl. Math., 2:482 [1981]; Needleman and Wunsch, J. Mol. Biol., 48:443 [1970]; Pearson and Lipman, Proc. Natl. Acad. Sci. USA 85:2444 [1988]; programs such as GAP, BESTFIT, FASTA, and TFASTA in the Wisconsin Genetics Software Package (Genetics Computer Group, Madison, Wis.); and Devereux et al., Nucl. Acid Res., 12:387-395 [1984]).

For example, PILEUP is a useful program to determine sequence homology levels. PILEUP creates a multiple sequence alignment from a group of related sequences using progressive, pairwise alignments. It can also plot a tree showing the clustering relationships used to create the alignment. PILEUP uses a simplification of the progressive alignment method of Feng and Doolittle, (Feng and Doolittle, J. Mol. Evol., 35:351-360 [1987]). The method is similar to that described by Higgins and Sharp (Higgins and Sharp, CABIOS 5:151-153 [1989]). Useful PILEUP parameters including a default gap weight of 3.00, a default gap length weight of 0.10, and weighted end gaps. Another example of a useful algorithm is the BLAST algorithm, described by Altschul et al., (Altschul et al., J. Mol. Biol., 215:403-410, [1990]; and Karlin et al., Proc. Natl. Acad. Sci. USA 90:5873-5787 [1993]). One particularly useful BLAST program is the WU-BLAST-2 program (See, Altschul et al., Meth. Enzymol., 266:460-480 [1996]). parameters “W,” “T,” and “X” determine the sensitivity and speed of the alignment. The BLAST program uses as defaults a wordlength (W) of 11, the BLOSUM62 scoring matrix (See, Henikoff and Henikoff, Proc. Natl. Acad. Sci. USA 89:10915 [1989]) alignments (B) of 50, expectation (E) of 10, M′5, N′4, and a comparison of both strands.

As used herein, “percent (%) nucleic acid sequence identity” is defmed as the percentage of nucleotide residues in a candidate sequence that are identical with the nucleotide residues of the sequence.

As used herein, the term “hybridization” refers to the process by which a strand of nucleic acid joins with a complementary strand through base pairing, as known in the art.

As used herein, “maximum stringency” refers to the level of hybridization that typically occurs at about Tm-5° C. (5° C. below the Tm of the probe); “high stringency” at about 5° C. to 10° C. below Tm; “intermediate stringency” at about 10° C. to 20° C. below Tm; and “low stringency” at about 20° C. to 25° C. below Tm. As will be understood by those of skill in the art, a maximum stringency hybridization can be used to identify or detect identical polynucleotide sequences while an intermediate or low stringency hybridization can be used to identify or detect polynucleotide sequence homologs.

The phrases “substantially similar and “substantially identical” in the context of two nucleic acids or polypeptides typically means that a polynucleotide or polypeptide comprises a sequence that has at least 75% sequence identity, preferably at least 80%, more preferably at least 90%, still more preferably 95%, most preferably 97%, sometimes as much as 98% and 99% sequence identity, compared to the reference (i.e., wild-type) sequence. Sequence identity may be determined using known programs such as BLAST, ALIGN, and CLUSTAL using standard parameters. (See e.g., Altschul, et al., J. Mol. Biol., 215:403-410 [1990]; Henikoff et al., Proc. Natl. Acad. Sci. USA 89:10915 [1989]; Karin et al., Proc. Natl. Acad. Sci. USA 90:5873 [1993]; and Higgins et al., Gene 73:237-244 [1988]). Software for performing BLAST analyses is publicly available through the National Center for Biotechnology Information. Also, databases may be searched using FASTA (Pearson et al., Proc. Natl. Acad. Sci. USA 85:2444-2448 [1988]).

As used herein, “equivalent residues” refers to proteins that share particular amino acid residues. For example, equivalent resides may be identified by determining homology at the level of tertiary structure for a protein (e.g. IFN-β) whose tertiary structure has been determined by x-ray crystallography. Equivalent residues are defined as those for which the atomic coordinates of two or more of the main chain atoms of a particular amino acid residue of the protein having putative equivalent residues and the protein of interest (N on N, CA on CA, C on C and O on O) are within 0.13 nm and preferably 0.1 nm after alignment. Alignment is achieved after the best model has been oriented and positioned to give the maximum overlap of atomic coordinates of non-hydrogen protein atoms of the proteins analyzed. The preferred model is the crystallographic model giving the lowest R factor for experimental diffraction data at the highest resolution available, determined using methods known to those skilled in the art of crystallography and protein characterization/analysis.

In some embodiments, modification is preferably made to the “precursor DNA sequence” which encodes the amino acid sequence of the precursor enzyme, but can be by the manipulation of the precursor protein. In the case of residues which are not conserved, the replacement of one or more amino acids is limited to substitutions which produce a variant which has an amino acid sequence that does not correspond to one found in nature. In the case of conserved residues, such replacements should not result in a naturally-occurring sequence. Derivatives provided by the present invention further include chemical modification(s) that change the characteristics of the protease.

In some preferred embodiments, the protein gene is ligated into an appropriate expression plasmid. The cloned protein gene is then used to transform or transfect a host cell in order to express the protein gene. This plasmid may replicate in hosts in the sense that it contains the well-known elements necessary for plasmid replication or the plasmid may be designed to integrate into the host chromosome. The necessary elements are provided for efficient gene expression (e.g., a promoter operably linked to the gene of interest). In some embodiments, these necessary elements are supplied as the gene's own homologous promoter if it is recognized, (i.e., transcribed, by the host), a transcription terminator (a polyadenylation region for eukaryotic host cells) which is exogenous or is supplied by the endogenous terminator region of the protein gene. In some embodiments, a selection gene such as an antibiotic resistance gene that enables continuous cultural maintenance of plasmid-infected host cells by growth in antimicrobial-containing media is also included.

The present invention encompasses proteins having altered immunogenicity that are equivalent. Being “equivalent,” means that the proteins are encoded by a polynucleotide capable of hybridizing to the polynucleotide having the sequence as shown in any one of those provided herein, under conditions of medium to high stringency and still retaining the altered immunogenic response to human T-cells. Being “equivalent” means that the protease comprises at least 55%, at least 65%, at least 70%, at least 75%, at least 80%, at least 85%, at least 90%, at least 95%, at least 97% or at least 99% identity to the epitope sequences and the variant proteases having such epitopes (e.g., having the amino acid sequence modified).

As used herein, the terms “hybrid proteins” and “fusion proteins ” refer to proteins 10 that are engineered from at least two different or “parental” proteins. In preferred embodiments, these parental proteins are homologs of one another. For example, in some embodiments, a preferred hybrid protease or fusion protein contains the N-terminus of a protein and the C-terminus of a homolog of the protein. In some preferred embodiment, the two terminal ends are combined to correspond to the full-length active protein. In alternative preferred embodiments, the homologs share substantial similarity but do not have identical T-cell epitopes. Therefore, in one embodiment, the present invention provides a protease of interest having one or more T-cell epitopes in the C-terminus, but in which the C-terminus is replaced with the C-terminus of a homolog having a less potent T-cell epitope, or fewer or no T-cell epitopes in the C-terminus. Thus, the skilled artisan understands that by being able to identify T-cell epitopes among homologs, a variety of variants producing different immunogenic responses can-be formed. Moreover, it is understood that internal portions, and more than one homolog can be used to produce the variants of the present invention.

“Operably linked” and “in operable combination,” when describing the relationship between two DNA regions, simply means that they are functionally related to each other. For example, a presequence is operably linked to a peptide if it functions as a signal sequence, participating in the secretion of the mature form of the protein most probably involving cleavage of the signal sequence. A promoter is operably linked to a coding sequence if it controls the transcription of the sequence; a ribosome binding site is operably linked to a coding sequence if it is positioned so as to permit translation.

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotides is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also may be said to have 5′ and 3′ ends. In either a linear or circular DNA s molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct transcription of a linked gene are generally located 5′ or upstream of the coding region (enhancer elements can exert their effect even when located 3′ of the promoter element and the coding region). Transcription termination and polyadenylation signals are located 3′ or downstream of the coding region.

The term “an oligonucleotide having a nucleotide sequence encoding a gene” means a DNA sequence comprising the coding region of a gene or, in other words, the DNA sequence that encodes a gene product. The coding region may be present in either a cDNA or genomic DNA form. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

The term “recombinant oligonucleotide” refers to an oligonucleotide created using molecular biological manipulations, including but not limited to, the ligation of two or more oligonucleotide sequences generated by restriction enzyme digestion of a polynucleotide sequence, the synthesis of oligonucleotides (e.g., the synthesis of primers or oligonucleotides) and the like.

The term “transcription unit” as used herein refers to the segment of DNA between the sites of initiation and termination of transcription and the regulatory elements necessary for the efficient initiation and termination. For example, a segment of DNA comprising an enhancer/promoter, a coding region, and a termination and polyadenylation sequence comprises a transcription unit.

The term “regulatory element” as used herein refers to a genetic element that controls some aspect of the expression of nucleic acid sequences. For example, a promoter is a regulatory element which facilitates the initiation of transcription of an operably linked coding region. Other regulatory elements are splicing signals, polyadenylation signals, termination signals, etc. (defined infra).

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals. Once transformed into a suitable host, the vector may replicate and function independently of the host genome, or may, in some instances, integrate into the genome itself. In the present specification, “plasmid” and “vector” are sometimes used interchangeably as the plasmid is the most commonly used form of vector at present. However, the invention is intended to include such other forms of expression vectors which serve equivalent functions and which are, or become, known in the art, including but not limited to plasmids, phage particles, viral vectors or simply potential genomic inserts.

The “host cells” used in the present invention generally are prokaryotic or eukaryotic hosts which contain an expression vector and/or gene of interest. Host cells are transformed or transfected with vectors constructed using recombinant DNA techniques. Such transformed host cells are capable of either replicating vectors encoding the protein variants or expressing the desired protein variant. In the case of vectors which encode the pre- or prepro-form of the protein variant, such variants, when expressed, are typically secreted from the host cell into the host cell medium.

The term “promoter/enhancer” denotes a segment of DNA which contains sequences capable of providing both promoter and enhancer functions (for example, the long terminal repeats of retroviruses contain both promoter and enhancer functions). The enhancer/promoter may be “endogenous” or “exogenous” or “heterologous.” An endogenous enhancer/promoter is one which is naturally linked with a given gene in the genome. An exogenous (heterologous) enhancer/promoter is one which is placed in juxtaposition to a gene by means of genetic manipulation (i.e., molecular biological techniques).

The presence of “splicing signals” on an expression vector often results in higher levels of expression of the recombinant transcript. Splicing signals mediate the removal of introns from the primary RNA transcript and consist of a splice donor and acceptor site (Sambrook et al., Molecular Cloning: A Laboratory Manual, 2nd ed., Cold Spring Harbor Laboratory Press, New York [1989], pp. 16.7-16.8). A commonly used splice donor and acceptor site is the splice junction from the 16S RNA of SV40.

Efficient expression of recombinant DNA sequences in eukaryotic cells requires signals directing the efficient termination and polyadenylation of the resulting transcript. Transcription termination signals are generally found downstream of the polyadenylation signal and are a few hundred nucleotides in length. The temp “poly A site” or “poly A sequence” as used herein denotes a DNA sequence which directs both the termination and polyadenylation of the nascent RNA transcript. Efficient polyadenylation of the recombinant transcript is desirable as transcripts lacking a poly A tail are unstable and are rapidly degraded. The poly A signal utilized in an expression vector may be “heterologous” or “endogenous.” An endogenous poly A signal is one that is found naturally at the 3′ end of the coding region of a given gene in the genome. A heterologous poly A signal is one which is isolated from one gene and placed 3′ of another gene. A commonly used heterologous poly A signal is the SV40 poly A signal.

The terms “stable transfection” and “stably transfected” refer to the introduction and integration of foreign DNA into the genome of the transfected cell. The term “stable transfectant” refers to a cell which has stably integrated foreign DNA into the genomic DNA.

The terms “selectable marker” and “selectable gene product” as used herein refer to the use of a gene which encodes an enzymatic activity that confers resistance to an antibiotic or drug upon the cell in which the selectable marker is expressed.

As used herein, the terms “amplification” and “gene amplification” refer to a process by which specific DNA sequences are disproportionately replicated such that the amplified gene becomes present in a higher copy number than was initially present in the genome. In some embodiments, selection of cells by growth in the presence of a drug (e.g., an inhibitor of an inhibitable enzyme) results in the amplification of either the endogenous gene encoding the gene product required for growth in the presence of the drug or by amplification of exogenous (i.e., input) sequences encoding this gene product, or both. Gene amplification occurs naturally during development in particular genes such as the amplification of ribosomal genes in amphibian oocytes. Gene amplification may be induced by treating cultured cells with drugs. An example of drug-induced amplification is the methotrexate-induced amplification of the endogenous dhfr gene in mammalian cells (Schmike et al., Science 202:1051 [1978]). Selection of cells by growth in the presence of a drug (e.g., an inhibitor of an inhibitable enzyme) may result in the amplification of either the endogenous gene encoding the gene product required for growth in the presence of the drug or by amplification of exogenous (i.e., input) sequences encoding this gene product, or both.

Amplification is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

As used herein, the term “co-amplification” refers to the introduction into a single cell of an amplifiable marker in conjunction with other gene sequences (i.e., comprising one or more non-selectable genes such as those contained within an expression vector) and the application of appropriate selective pressure such that the cell amplifies both the amplifiable marker and the other, non-selectable gene sequences. The amplifiable marker may be physically linked to the other gene sequences or alternatively two separate pieces of DNA, one containing the amplifiable marker and the other containing the non-selectable marker, may be introduced into the same cell.

As used herein, the terms “amplifiable marker,” “amplifiable gene,” and “amplification vector” refer to a gene or a vector encoding a gene which permits the amplification of that gene under appropriate growth conditions.

As used herein, the term “amplifiable nucleic acid” refers to nucleic acids which may be amplified by any amplification method. It is contemplated that “amplifiable nucleic acid” will usually comprise “sample template.”

As used herein, the term “sample template” refers to nucleic acid originating from a sample which is analyzed for the presence of “target” (defined below). In contrast, “background template” is used in reference to nucleic acid other than sample template which may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

“Template specificity” is achieved in most amplification techniques by the choice of enzyme. Amplification enzymes are enzymes that, under conditions they are used, will process only specific sequences of nucleic acid in a heterogeneous mixture of nucleic acid. For example, in the case of Qβ replicase, MDV-1 RNA is the specific template for the replicase (See e.g., Kacian et al., Proc. Natl. Acad. Sci. USA 69:303S [1972]). Other nucleic acids are not replicated by this amplification enzyme. Similarly, in the case of T7 RNA polymerase, this amplification enzyme has a stringent specificity for its own promoters (See, Chamberlin et al., Nature 228:227 [1970]). In the case of T4 DNA ligase, the enzyme will not ligate the two oligonucleotides or polynucleotides, where there is a mismatch between the oligonucleotide or polynucleotide substrate and the template at the ligation junction (See, Wu and Wallace, Genomics 4:560 [1989]). Finally, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, which is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the term “target,” when used in reference to the polymerase chain reaction, refers to the region of nucleic acid bounded by the primers used for polymerase chain reaction. Thus, the “target” is sought to be sorted out from other nucleic acid sequences. A “segment” is defined as a region of nucleic acid within the target sequence.

As used herein, the term “polymerase chain reaction” (“PCR”) refers to the methods of U.S. Pat. Nos. 4,683,195 4,683,202, and 4,965,188, hereby incorporated by reference, which include methods for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”.

As used herein, the term “amplification reagents” refers to those reagents (deoxyribonucleotide triphosphates, buffer, etc.), needed for amplification except for primers, nucleic acid template and the amplification enzyme. Typically, amplification reagents along with other reaction components are placed and contained in a reaction vessel (test tube, microwell, etc.).

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein, the terms “restriction endonucleases” and “restriction enzymes” refer to bacterial enzymes, each of which cut double-stranded DNA at or near a specific nucleotide sequence.

The terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

The peptides of the present invention and pharmaceutical and vaccine compositions thereof are useful for administration to mammals, particularly humans, to treat and/or prevent HPV infection. Vaccines that contain an immunogenically effective amount of one or more peptides as described herein are a further embodiment of the invention. Once appropriately immunogenic epitopes have been defined, they can be delivered by various means, herein referred to as “vaccine” compositions. Such vaccine compositions can include, for example, lipopeptides (e.g., Vitiello et al., J Clin. Invest., 95:341 [1995]; and PCTUSOO/17842; peptide compositions encapsulated in poly(DL-lactide-co-glycolide) (“TLG”) microspheres (See e.g., Eldridge et al., Molec. Immunol., 28:287-294 [1991]: Alonso et al., Vaccine 12:299-306 [1994]; Jones et al., Vaccine 13:675-681 [1995]), peptide compositions contained in immune stimulating complexes (ISCOMS) (See e.g., Takahashi et al., Nature 344:873-875 [1990]; Hu et al., Clin. Exp. Immunol., 113:235-243 [1998]), multiple antigen peptide systems (MAPs) (See e.g., Tam, Proc. Natl. Acad. Sci. U.S.A. 85:5409-5413 [1988]; Tam, J. Immunol. Meth., 196:17-32 [1996]), viral delivery vectors (Perkus et al, In: Concepts in Vaccine Development, Kaufinann (ed.), p. 379 [1996]; Chakrabarti et al., Nature 320:535 [1986]; Hu et al., Nature 320:537 [1986]; Kieny et al., AIDS Bio/Technol., 4:790 [1986]; Top et al., J Infect. Dis., 124:148 [1971]; Chanda et al., Virol.,175:535 [1990]), particles of viral or synthetic origin (e.g., Kofler et al., J Immunol., Meth., 192:25 [1996]; Eldridge et al., Sem. Hematol., 30:16 [1993]; Falo et al., Nature Med., 7:649 [1995]), adjuvants (Warren et al., Ann. Rev. Immunol., 4:369 [1986]; Gupta et al., Vaccine 11:293 [1993]), liposomes (Reddy et al., J Immunol., 148:1585 [1992]; Rock, Immunol. Today 17:131 [1996]), or, naked or particle absorbed cDNA (Ulmer et al., Science 259:1745 [1993]; Robinson et al., Vaccine 11:957 [1993]; Shiver et al., In: Concepts in Vaccine Development, Kaufinann (ed), p. 423 [1996]; Cease and Berzofsky, Ann. Rev. Immunol., 12:923 [1994]; and Eldridge et al., Sem. Hematol., 30:16 [1993]). Toxin-targeted delivery technologies, also known as receptor mediated targeting, such as those of Avant Immunotherapeutics, Inc. (Needham, Mass.) may also be used.

Vaccine compositions of the invention include nucleic acid-mediated modalities. DNA or RNA encoding one or more of the peptides of the invention can also be administered to a patient. This approach is described, for instance, in Wolff et. al., Science 247:1465 (1990) as well as U.S. Pat. Nos. 5,580,859; 5,589,466; 5,804,566; 5,739,118; 5,736,524; 5,679,647; WO 98/04720; and in more detail below. Examples of DNA-based delivery technologies include “naked DNA,” facilitated (bupivicaine, polymers, peptide-mediated) delivery, cationic lipid complexes, and particle-mediated (“gene gun”) or pressure-mediated delivery (See e.g., U.S. Pat. No. 5,922,687).

For therapeutic or prophylactic immunization purposes, the peptides of the invention can be expressed by viral or bacterial vectors. Examples of expression vectors include attenuated viral hosts, such as vaccinia or fowl pox. This approach involves the use of vaccinia virus, for example, as a vector to express nucleotide sequences that encode the peptides of the invention. Upon introduction into an acutely or chronically infected host or into a non-infected host, the recombinant vaccinia virus expresses the immunogenic peptide, and thereby elicits a host CTL and/or HTL response. Vaccinia vectors and methods useful in immunization protocols are described in, e. g., U.S. Pat. No. 4,722,848. Another vector is BCG (Bacille Calmette Guerin). BCG vectors are described in Stover et al., Nature 351:456-460 (1991). A wide variety of other vectors useful for therapeutic administration or immunization of the peptides of the invention, e.g. adeno and adeno-associated virus vectors, retroviral vectors, Salmonella typhi vectors, detoxified anthrax toxin vectors, and the like, will be apparent to those skilled in the art from the description herein.

Furthermore, vaccines in accordance with the invention can encompass one or more of the peptides of the invention. Accordingly, a peptide can be present in a vaccine individually. Alternatively, the peptide can be individually linked to its own carrier; alternatively, the peptide can exist as a homopolymer comprising multiple copies of the same peptide, or as a heteropolymer of various peptides. Polymers have the advantage of increased immunological reaction and, where different peptide epitopes are used to make up the polymer, the additional ability to induce antibodies and/or CTLs that react with different antigenic determinants of the pathogenic organism targeted for an immune response. The composition may be a naturally occurring region of an antigen or may be prepared, e.g., recombinantly or by chemical synthesis.

Carriers that can be used with vaccines of the invention are well known in the art, and include, e.g., thyroglobulin, albumins such as human serum albumin, tetanus toxoid, polyamino acids such as poly L-lysine, poly L-glutamic acid, influenza, hepatitis B virus core protein, and the like. The vaccines can contain a physiologically tolerable (i.e., acceptable) diluent such as water, or saline, preferably phosphate buffered saline. The vaccines also typically include an adjuvant. Adjuvants such as incomplete Freund's adjuvant, aluminum phosphate, aluminum hydroxide, or alum are examples of materials well known in the art. Additionally, CTL responses can be primed by conjugating peptides of the invention to lipids, such as tiipalmitoyl-S-glycerylcysteinlyseryl-serine (P3CSS).

Upon immunization with a peptide composition in accordance with the invention, via injection, aerosol, oral, transdermal, transmucosal, intrapleural, intrathecal, or other suitable routes, the immune system of the host responds to the vaccine by initiating a CD4⁺ T-cell response.

Consequently, the host becomes at least partially immune to later infection, or at least partially resistant to developing an ongoing chronic infection, or derives at least some therapeutic benefit when the antigen was tumor-associated.

In certain embodiments, components that induce T-cell responses are combined with component that induce antibody responses to the target antigen of interest. A preferred embodiment of such a composition comprises Class I and Class II epitopes in accordance with the invention.

For pharmaceutical compositions, the immunogenic peptides of the invention are administered to an individual already infected with HPV. Those in the incubation phase or the acute phase of infection can be treated with the immunogenic peptides separately or in conjunction with other treatments, as appropriate. In therapeutic applications, compositions are administered to a patient in an amount sufficient to elicit an effective CD4⁺ T-cell response to the virus and to cure or at least partially arrest symptoms and/or complications. An amount adequate to accomplish this is defined as “therapeutically effective dose.” Amounts effective for this use will depend on, e.g., the peptide composition, the manner of administration, the stage and severity of the disease being treated, the weight and general state of health of the patient, and the judgment of the prescribing physician, but generally range for the initial immunization (that is for therapeutic or prophylactic administration) from about 1.0 ug to about 50,000 ug of peptide for a 70 kg patient, followed by boosting dosages of from about 1.0 ug to about 10,000 ug of peptide pursuant to a boosting regimen over weeks to months depending upon the patient's response and condition by measuring specific CD4⁺ T-cell activity in the patient's blood.

Immunizing doses followed by boosting doses at established intervals, e. g., from one to four weeks, may be required, possibly for a prolonged period of time to effectively immunize an individual. In the case of chronic infection, administration should continue until at least clinical symptoms or laboratory tests indicate that the viral infection has been eliminated or substantially abated and for a period thereafter.

The pharmaceutical compositions for therapeutic treatment are intended for parenteral, topical, oral or local administration. Preferably, the pharmaceutical compositions are administered parenterally, e.g., intravenously, subcutaneously, intradermally, or intramuscularly. Thus, the invention provides compositions for parenteral administration which comprise a solution of the immunogenic peptides dissolved or suspended in an acceptable carrier, preferably an aqueous carrier. A variety of aqueous carriers may be used, e.g., water, buffered water, 0.9% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolarnine oleate, etc.

### DETAILED DESCRIPTION OF THE INVENTION

The present invention provides CD4+ T-cell epitopes in E6, E7 and E2 proteins from various strains of human papillomavirus (HPV). In some preferred embodiments, the present invention provides means for the development of HPV vaccines, in particular multivalent vaccines for the prevention of infection with high-risk HPV strains. In additional embodiments, the present invention provides means for the development of therapeutic vaccines against high-risk HPV types suitable for use in the prevention of the development of benign and/or malignant tumors in infected individuals. The present invention further provides epitopes suitable for use in prophylactic and/or therapeutic vaccines. In particularly preferred embodiments, the present invention provides modified epitopes suitable for use in prophylactic and/or therapeutic vaccines.

E6 and E7 oncoproteins from HPV represent especially attractive targets for a DNA vaccine because of their ubiquitous expression in cervical carcinoma cases. E6 and E7 proteins are responsible for the oncogenic characteristics of HPV (Finzer et al., Cancer Lett., 188:15-24 [2002]). Continued expression of these two proteins is necessary for continued proliferation and survival of cervical cancer cells (von Knebel-Doeberitz et al., Cancer Res., 48:3780-6 [1988]). E6 and E7 are responsible for transformation in cervical lesions and inhibition of apoptosis. Several studies have indicated that immunological responses against these proteins can be protective against cervical cancer. Natural CTL responses to E6 and E7 have been shown to be more common in HPV16 positive women without squamous intraepithelial neoplasia (SIL) than in HPV 16 positive women with SIL (Nakagawa et al., J. Infect. Dis., 175: 927-931 [1997]). In addition, cell-mediated immune responses to specific protective peptides of E6 and E7 have been correlated with disease regression and resolution of viral infection (Kadish et al., Cancer Epidemiol. Biomarkers Prev., 11:483-488 [2002]). Vaccination with E7 DNA has been demonstrated to be highly efficient at eliciting cytotoxic T-cell response (Osen et al., Vaccine 19:4276-4286 [2001]).

E2 is also a potential epitope useful in vaccines. Indeed, it is contemplated that CIN (cervical intra-epithelial neoplasia) grade I (i.e., only ⅓ of the thickness of the surface layer of the cervix is affected), may be better targeted via E2 and/or E1 epitopes than other HPV epitopes, as CIN is an early precursor to cervical cancer. In CIN I, E2 expression is greatest, while it is down-regulated in CIN III lesions (i.e., the full thickness of the cervical surface layer is affected; also referred to as “carcinoma-in-situ,” although it is not cervical cancer) (See e.g., Torng et al., J. Surg. Oncol., 84:17-23 [2003]; Stevenson et al., J. Gen. Virol., 81:1825-1832 [2000]; and Maitland et al., J. Pathol., 186:275-280 [1998]). The expression pattern of E2 in early infection and low-grade CIN indicates that E2 is a good vaccine candidate for pre-cancerous lesions where E6 and E7 expression is low. Additionally, functional domains of E2 proteins are highly conserved and it is contemplated that inclusion of E2 epitopes in vaccine constructs will find use in inducing immunity across several HPV strains. In addition, it has been observed that inclusion of E2 epitopes improves HLA population coverage and epitope redundancy. Furthermore, more than 50% of healthy subjects have been shown to have a strong T-helper cell memory response against E2 and E6 epitopes (See e.g., Welters et al., Cancer Res., 63:636-641 [2003]; and de Jong et al., Cancer Res., 62:472-479 [2002]). Thus, it is contemplated that E2 will find use as a component of many HPV vaccine compositions.

The present invention, in which an epitope vaccine is used rather than a full-length vaccine, is attractive because it obviates the concern of administering an oncogenic product. Also, because of size constraints of a DNA vaccine, inclusion of only immunogenic regions of E6 and E7 allows for the coverage of,more high risk strains. Patients with HPV infections often carry more than one HPV strain, and individuals who clear an HPV infection of one strain can become re-infected with a second strain. Although CTL epitopes are typically associated with antiviral vaccines, there are several reasons for including CD4+ epitopes in some preferred embodiments of the present invention. For example, antigen-specific CD4 help is generally required for activation of CD8 cytolytic activity through dross-priming of antigen presenting cells (See, Bennett et al., Nature 393:478-480 [1998]; Schoenberger et al., Nature 393:480-483 [1998]; and Ridge et al., Nature 393:474-478 [1998]). Furthermore, studies in animal models have demonstrated that vaccines that include both CD4 and CD8 epitopes derived from the same antigen induce a strong protective response (See, Ossendrop et al., J. Exp. Med., 187:693-702 [1998]; De Veermann et al., J. Immunol., 162: 144-151 [1999]; and Zwaveling et al, J. Immunol., 169:350-8 [2002]). Additionally, although CD8 positive T-cells are known to be the primary effector cells in protection against viral infection, some studies have demonstrated the direct effect of CD4 cells in mediating cytolytic activity (See, Bourgault et al., J. Immunol., 142:252-256 [1989]; Khanna et al., J. Immunol., 158:3619-3625 [1997]). In additional studies, CD4 T lymphocytes have been demonstrated to directly contribute to HPV16 E6 and E7 CTL activity (Altmann et al., Eur. J. Cancer 128:326-33 [1992]; and Nakagawa et al., Clin. Diag. Lab. Immunol., 6:494-498 [1999).

The present invention provides embodiments encompassing compositions and methods for the development of vaccine compositions directed against the E6 and E7 proteins of four high risk HPV strains (i.e., strains 16, 18, 45 and 56) and four moderate-risk HPV strains (i.e., strains 31, 33, 52 and 58. Importantly, the presence of DNA from these HPV strains has been associated with cervical lesions and cancers (Lorincz et al., Obstet. Gynecol., 79:328-337. [1992]). Using a CD4 T-cell proliferation assay (Genencor's proprietary I-MUNE® assay), HLA class II helper epitopes in E6 and E7 proteins of each of the above mentioned strains of HPV were identified. It is contemplated that the compositions and methods of the present invention will find use therapeutic and/or preventative vaccine compositions.

In addition to the E6 and E7 epitopes, E2 epitopes are also provide herein. As with the E6 and E7 epitopes, the E2 epitopes of four HPV strains were identified using the I-MUNE® assay system. It is contemplated that the compositions and methods of the present invention will find use therapeutic and/or preventative vaccine compositions.

Experimental

The following examples are provided in order to demonstrate and further illustrate certain preferred embodiments and aspects of the present invention and are not to be construed as limiting the scope thereof.

In the experimental disclosure which follows, the following abbreviations apply: M (molar); mM (millimolar); μM (micromolar); nM (nanomolar); mol (moles); mmol (millimoles); μmol (micromoles); nmol (nanomoles); gm (grams); mg (milligrams); μg (micrograms); pg (picograms); L (liters); ml (milliliters); μl (microliters); cm (centimeters); mm (millimeters); μm (micrometers); nm (nanometers); ° C. (degrees Centigrade); cDNA (copy or complimentary DNA); DNA (deoxyribonucleic acid); ssDNA (single stranded DNA); dsDNA (double stranded DNA); dNTP (deoxyribonucleotide triphosphate); RNA (ribonucleic acid); PBS (phosphate buffered saline); g (gravity); OD (optical density); Dulbecco's phosphate buffered solution (DPBS); HEPES (N-[2-Hydroxyethyl]piperazine-N-[2-ethanesulfonic acid]); HBS (HEPES buffered saline); SDS (sodium dodecylsulfate); Tris-HCl (tris[Hydroxymethyl]aminomethane-hydrochloride); DMSO (dimethyl sulfoxide); EGTA (ethylene glycol-bis(β-aminoethyl ether) N,N,N′,N′-tetraacetic acid); EDTA (ethylenediaminetetracetic acid); DPBS (Dulbecco's phosphate buffered solution); bla (β-lactamase or ampicillin-resistance gene); Endogen (Endogen, Woburn, Mass.); CytoVax (CytoVax, Edmonton, Canada); Wyeth-Ayerst (Wyeth-Ayerst, Philadelphia, Pa.); NEN (NEN Life Science Products, Boston, Mass.); Wallace Oy (Wallace Oy, Turku, Finland); Pharma AS (Pharma AS, Oslo, Norway); Dynal (Dynal, Oslo, Norway); Bio-Synthesis (Bio-Synthesis, Lewisville, Tex.); Mimotopes (Mimotopes, Inc., San Diego, Calif.); ATCC (American Type Culture Collection, Rockville, Md.); Gibco/BRL (Gibco/BRL, Grand Island, N.Y.); Sigma (Sigma Chemical Co., St. Louis, Mo.); Pharmacia (Pharmacia Biotech, Piscataway, N.J.); Invitrogen (Invitrogen, Inc., Grand Island, N.Y.); Abbott (Abbott Laboratories, Abbott Park, Ill.); List (List Biological Laboratories Inc., Campbell, Calif.); Perkin Elmer (Perkin Elmer Life Sciences, Boston Mass.); and Stratagene (Stratagene, La Jolla, Calif.).

### EXAMPLE 1

### Preparation of E6, E7 and E2 Epitopes

Full length amino acid sequences of E6 and E7 proteins from HPV 16, 18, 31, 33, 45, 52, 56 and 58 were used to create 15-mer peptide sets. SwissProt P03126 corresponds to HPV16 E6, while SwissProt. P03129 corresponds to HPV16 E7. SwissProt. P06463 corresponds to HPV18 E6, while SwissProt. P06788 corresponds to HPV18 E7. SwissProt. P17386 corresponds to HPV31 E6, while SwissProt. P17387 corresponds to HPV31 E7. SwissProt. P06427 corresponds to HPV33 E6, while SwissProt. P06429 corresponds to HPV33 E7. SwissProt. P21735 corresponds to HPV45 E6, while SwissProt. P21736 corresponds to HPV45 E7. SwissProt. P36814 corresponds to HPV52 E6, while SwissProt. P36831 corresponds to HPV52 E7. SwissProt. P24836 corresponds to HPV56 E6, while SwissProt. P36833 corresponds to HPV56 E7. SwissProt. P26555 corresponds to HPV58 E6, while SwissProt. P26557 corresponds to HPV58 E7. SwissProt. P03120 corresponds to HPV16 E2, while SwissProt. P06790 corresponds to HPV18 E2, SwissProt. P17383 corresponds to HPV31 EP2, SwissProt. P06423 corresponds to HPV33 E2, SwissProt. P36794 corresponds to HPV45 E2, SwissProt. P36796 corresponds to HPV52 E2, SwissProt. P36798 corresponds to HPV56 E2, and SwissProt. P26546 corresponds to HPV58 E2.

These variant peptides were synthesized by Mimotopes, using the multi-pin synthesis technique known in the art (See e.g., Maeji et al., J. Immunol. Meth., 134:23-33 [1990]). The 15-mer peptides were created such that sequences with adjacent peptides shared 12 amino acids (i.e., each peptide was offset by three amino acids). Peptides were diluted with DMSO to provide a stock concentration of approximately 2 mg/ml. The final concentration of peptides used in each assay was 5 μg/ml.

### EXAMPLE 2

### Preparation of Cells Used in the Assay System for the Identification of Peptide T-Cell Epitopes in HPV Using Human T-Cells

Fresh human peripheral blood cells were collected from humans of unknown exposure status to HPV. These cells were tested to determine antigenic epitopes in HPV 16, 18, 31, 33, 45, 52, 56, and 58, as described in Example 3.

Peripheral mononuclear blood cells (stored at room temperature, no older than 24 hours) were prepared for use as follows. PBMC's were isolated from buffy coat material by centrifuging over an underlay of Lymphoprep at 1000×g for 30 minutes. The interface layer was collected and washed and counted using the Cell-Dyn 3700 System (Abbott). Then, suspensions containing 10⁸ PBMC's resuspended in 30 ml of AIM-V (Invitrogen) were prepared and then allowed to adhere to plastic T-75 culture flasks for two hours. The remainder of the cells were frozen at 5×10⁷ cells/ml in 90% FCS (Gibco/BRL) and 10% DMSO (Sigma). After the two hour PBMC incubation, non-adherent cells were removed from the flasks. The adherent cells were cultured in the flasks with 800 units/ml recombinant human GM-CSF (Endogen) and 100 units/ml recombinant human IL-4 (Endogen) at 37° C., 5% CO₂. On day 5 of incubation, 50 units/ml recombinant human II-1α (Endogen) and 0.2 units/ml recombinant human TNF-α were added to the cultures. Adherent and non-adherent dendritic cells were harvested, washed, and counted on day 7, following a one-hour treatment with 30 mg/ml mitomycin C (Sigma) and 10 mM EDTA.

Autologous CD4+ T-cells were prepared from frozen aliquots of PBMCs. After thawing and washing in DPBS, CD4+ T-cells were isolated using a commercially available CD4 negative selection kit (Dynal), according to the manufacturer's instructions. Cells were counted using the Abbott Cell-Dyn 3700 System. The purity obtained using these methods was generally found to be greater than 90%.

### EXAMPLE 3

### T-Cell Proliferation Assays

This Example describes the assay system used in the present invention. This test s system is also referred to as the “I-MUNE®” assay system. In 96-well, round bottom plates, autologous dendritic cells and CD4+ T-cells were combined with test peptides. More specifically, in a volume of 100 μl/well, 2×10⁴ dendritic cells in AIM V were combined with individual peptides (at a final peptide concentration of 5 μg/ml and a final DMSO concentration of 0.25%). After a one-hour incubation at 37° C., 5% CO₂, 2×10⁵ CD4+ T-cells were added to the culture for a total volume of 200 μl. Negative control wells contained dendritic cells, CD4+ T-cells and 0.25% DMSO. Positive control wells contained dendritic cells, CD4+ T-cells (at the same concentrations as the test wells) and 0.25% DMSO with 0.4 μg/ml tetanus toxoid (List). Individual peptides were tested in duplicate for each donor.

After 5 days of incubation at 37° C., 5% CO₂, the cultures were pulsed with 0.25 μCi/well tritiated thymidine (Perkin Elmer). After a subsequent 24 hours of incubation, plates were harvested and assessed for incorporation of the tritiated thymidine (i.e., T-cell proliferation) using a Wallac Microbeta TriLux liquid scintillation counter (Perkin Elmer). Responses were averaged over the duplicate tests performed for each specimen. Positive responses were defined as having a response at least 2.95 times the background.

A set of data was accumulated for each of the proteins tested with at least 55 donors. The percent response rate for each peptide was determined for the entire population of donors. In this assay system, the “mean background response rate for a population of donors” is defined as the average percent response rate for all the peptides in a set. In this assay system, a “major epitope” is defined as having a response rate at least three standard deviations above the mean background response rate. “Moderate epitopes” are those epitopes that produce results that are at least two standard deviations above the mean or three times the background. “Minor epitopes” are those that have a response rate that is at least twice the background value. As described herein, this assay identified several epitopes in all of the HPV strains tested, with the exception of E7.33.

A. HPV E6.16

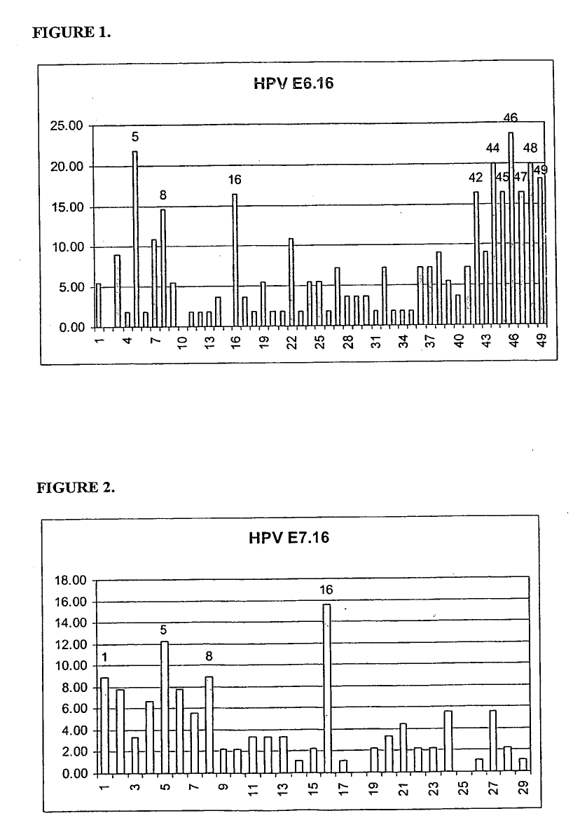

This antigen was tested with 55 donor samples in the I-MUNE® assay to determine epitopes for HPV E6.16. FIG. 1 provides a graph showing the responses for each of the epitope. As also indicated in Table 1, there were 10 epitopes of interest identified in this antigen.

TABLE 1
HPV E6.16 Epitopes of Interest

| Peptide | Epitope | | |
|---|---|---|---|
| Number | Classification | Epitope Sequence | SEQ ID NO: |
| 5 | Moderate | QERPRKLPQLCTELQ | SEQ ID NO: 1 |
| 8 | Minor | LCTELQTTIHDIILE | SEQ ID NO: 2 |
| 16 | Minor | RREVYDFAFRDLCIV | SEQ ID NO: 3 |
| 42 | Minor | RHLDKKQRFHNIRGR | SEQ ID NO: 4 |
| 44 | Moderate | QRFHNIRGRWTGRCM | SEQ ID NO: 5 |
| 45 | Minor | HNIRGRWTGRCMSCC | SEQ ID NO: 6 |
| 46 | Moderate | RGRWTGRCMSCCRSS | SEQ ID NO: 7 |
| 47 | Minor | WTGRCMSCCRSSRTR | SEQ ID NO: 8 |
| 48 | Moderate | RCMSCCRSSRTRRET | SEQ ID NO: 9 |
| 49 | Minor | MSCCRSSRTRRETQL | SEQ ID NO: 10 |

B. HPV E7.16

For this antigen, 90 donors were tested in the I-MUNE® assay to determine epitopes for HPV E7.16. FIG. 2 provides a graph showing the responses to each epitope. Also as indicated in Table 2, there were 4 epitopes of interest identified in this antigen.

TABLE 2
HPV E7.16 Epitopes of Interest

| Peptide | Epitope | | |
|---|---|---|---|
| Number | Classification | Epitope Sequence | SEQ ID NO: |
| 1 | Minor | MHGDTPTLHEYMLDL | SEQ ID NO: 11 |
| 5 | Moderate | LDLQPETTDLYCYEQ | SEQ ID NO: 12 |
| 8 | Minor | LYCYEQLNDSSEEED | SEQ ID NO: 13 |
| 16 | Moderate | EPDRAHYNIVTFCCK | SEQ ID NO: 14 |

C. HPV E6.18

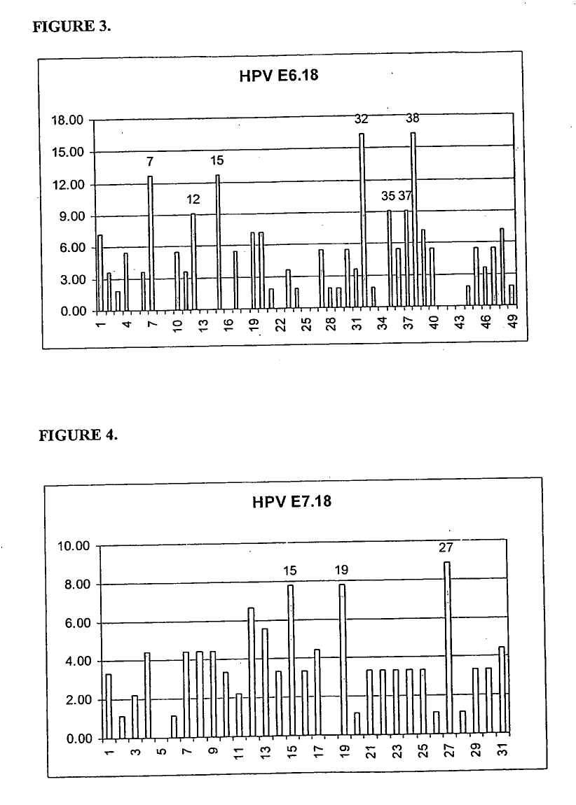

For this antigen, 55 donors were tested in the I-MUNE® assay to determine epitopes for HPV E6.18. FIG. 3 provides a graph showing the responses to each epitope. Also as indicated in Table 3, there were 7 epitopes of interest identified in this antigen.

TABLE 3
HPV E6.18 Epitopes of Interest

| Peptide | Epitope | | |
|---|---|---|---|
| Number | Classification | Epitope Sequence | SEQ ED NO: |
| 7 | Moderate | TELNTSLQDIEITCV | SEQ ID NO: 15 |
| 12 | Minor | YCKTVLELTEVFEFA | SEQ ID NO: 16 |
| 15 | Moderate | EVFEFAFKDLFVVYR | SEQ ID NO: 17 |
| 32 | Moderate | TNTGLYNLLIRCLRC | SEQ ID NO: 18 |
| 35 | Minor | IRCLRCQKPLNPAEK | SEQ ID NO: 19 |
| 37 | Minor | QKPLNPAEKLRHLNE | SEQ ID NO: 20 |
| 38 | Moderate | LNPAEKLRHLNEKRR | SEQ ID NO: 21 |

D. HPV E7.18

For this antigen, 90 donors were tested in the I-MUNE® assay to determine the epitopes of interest for HPV E7.18. FIG. 4 provides a graph showing the responses to each epitope. Also as indicated in Table 4, there were 3 epitopes of interest identified in this antigen.

TABLE 4
HPV E7.18 Epitopes of Interest

| Peptide | Epitope | | |
|---|---|---|---|
| Number | Classification | Epitope Sequence | SEQ ID NO: |
| 15 | Minor | GVNHQHLPARRAEPQ | SEQ ID NO: 22 |
| 19 | Minor | EPQRHTMLCMCCKCE | SEQ ID NO: 23 |
| 27 | Moderate | SADDLRAFQQLFLNT | SEQ ID NO: 24 |

E. HPV E6.31

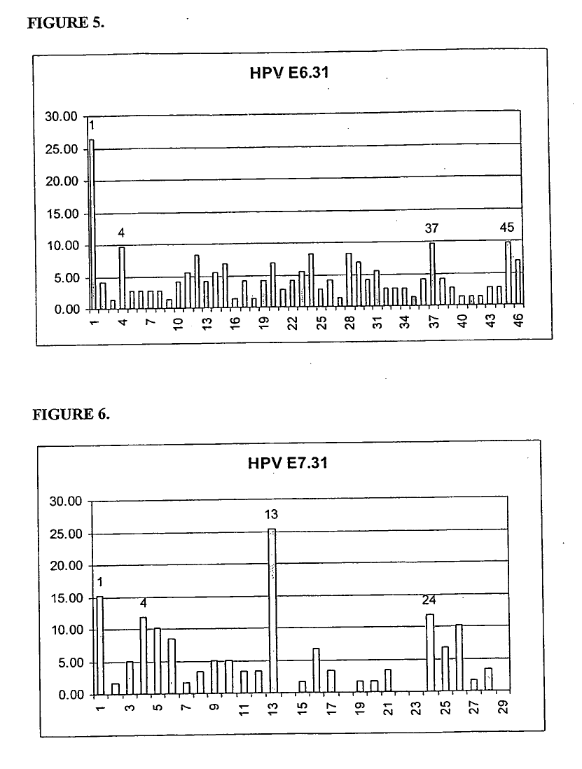

For this antigen, 72 donors were tested in the I-MUNE® assay to determine the epitopes of interest for HPV E6.31. FIG. 5 provides a graph showing the responses to each epitope. Also as indicated in Table 5, there were 4 epitopes of interest identified in this antigen.

TABLE 5
HPV E6.31 Epitopes of Interest

| Peptide | Epitope | | |
|---|---|---|---|
| Number | Classification | Epitope Sequence | SEQ ED NO: |
| 1 | Major | MFKNPAERPRKLHEL | SEQ ID NO: 25 |
| 4 | Minor | RKLHELSSALEIPYD | SEQ ID NO: 26 |
| 37 | Minor | PLCPEEKQRHLDKKK | SEQ ID NO: 27 |
| 45 | Minor | TGRCIACWRRPRTET | SEQ ID NO: 28 |

F. HPV E7.31

For this antigen, 59 donors were tested in the I-MUNE® assay to determine the epitopes of interest for HPV E7.31. FIG. 6 provides a graph showing the responses to each epitope. Also as indicated in Table 6, there were 4 epitopes of interest identified in this antigen.

TABLE 6
HPV E7.31 Epitopes of Interest

| Peptide | Epitope | | |
|---|---|---|---|
| Number | Classification | Epitope Sequence | SEQ ID NO: |
| 1 | Minor | MRGETPTLQDYVLDL | SEQ ID NO: 29 |
| 4 | Minor | DYVLDLQPEATDLHC | SEQ ID NO: 30 |
| 13 | Major | VIDSPAGQAEPDTSN | SEQ ID NO: 31 |
| 24 | Minor | QSTQVDIRILQELLM | SEQ ID NO: 32 |

G. HPV E6.33

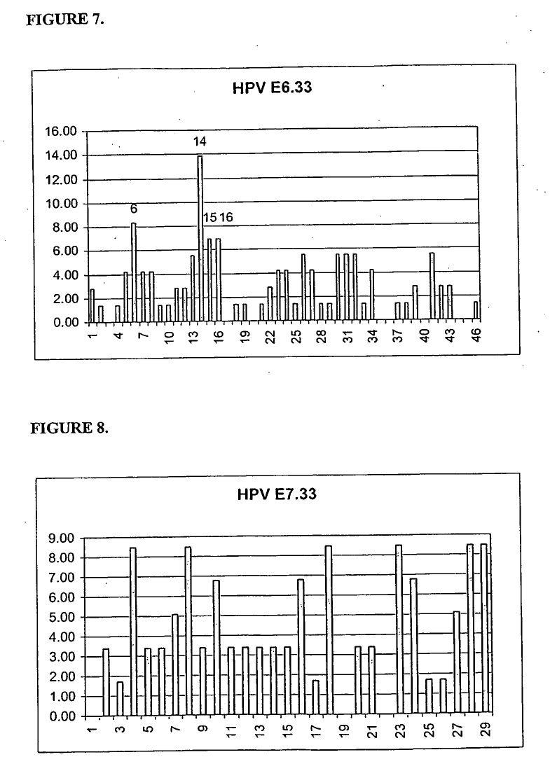

For this antigen, 72 donors were tested in the I-MUNE® assay to determine the epitopes of interest for HPV E6.33. FIG. 7 provides a graph showing the responses to each epitope. Also as indicated in Table 7, there were 4 epitopes of interest identified in this antigen.

TABLE 7
HPV E6.33 Epitopes of Interest

| Peptide | Epitope | | |
|---|---|---|---|
| Number | Classification | Epitope Sequence | SEQ ID NO: |
| 6 | Minor | CQALETTIHNIELQC | SEQ ID NO: 33 |
| 14 | Major | SEVYDFAFADLTVVY | SEQ ID NO: 34 |
| 15 | Minor | YDFAFADLTVVYREG | SEQ ID NO: 35 |
| 16 | Minor | AFADLTVVYREGNPF | SEQ ID NO: 36 |

H. HPV E733

For this antigen, 59 donors were tested in the I-MUNE® assay to determine the epitope of interest for HPV E7.33. FIG. 8 provides a graph showing the responses to each epitope. There were no epitopes of interest identified in this assay system.

I. HPV E6.45

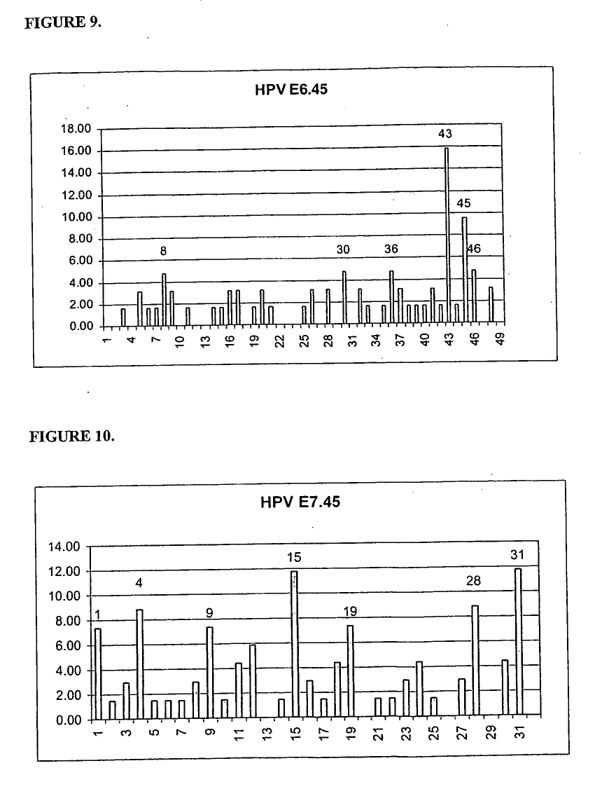

For this antigen, 63 donors were tested in the I-MUNE® assay to determine the epitopes of interest for HPV E6.45. FIG. 9 provides a graph showing the responses to each epitope. Also as indicated in Table 8, there were 6 epitopes of interest in this antigen.

TABLE 8
HPV E6.45 Epitopes of Interest

| Peptide | Epitope | | |
|---|---|---|---|
| Number | Classification | Epitope Sequence | SEQ ID NO: |
| 8 | Minor | NTSLQDVSIACVYCK | SEQ ID NO: 37 |
| 30 | Minor | ETLEKITNTELYNLL | SEQ ID NO: 38 |
| 36 | Minor | LRCQKPLNPAEKRRH | SEQ ID NO: 39 |
| 43 | Major | FHSIAGQYRGQCNTC | SEQ ID NO: 40 |
| 45 | Moderate | QYRGQCNTCCDQARQ | SEQ ID NO: 41 |
| 46 | Minor | GQCNTCCDQARQERL | SEQ ID NO: 42 |

J. HPV E7.45

For this antigen, 68 donors were tested in the I-MUNE® assay to determine the epitopes of interest for HPV E7.45. FIG. 10 provides a graph showing the responses to each epitope. Also as indicated in Table 9, there were 7 epitopes of interest identified in this antigen.

TABLE 9
HPV E7.45 Epitopes of Interest

| Peptide | Epitope | | |
|---|---|---|---|
| Number | Classification | Epitope Sequence | SEQ ID NO: |
| 1 | Minor | MHGPRETLQEIVLHL | SEQ ID NO: 43 |
| 4 | Minor | EIVLHLEPQNELDPV | SEQ ID NO: 44 |
| 9 | Minor | DLLCYEQLSESEEEN | SEQ ID NO: 45 |
| 15 | Moderate | DGVSHAQLPARRAEP | SEQ ID NO: 46 |
| 19 | Minor | AEPQRHKILCVCCKC | SEQ ID NO: 47 |
| 28 | Minor | EDLRTLQQLFLSTLS | SEQ ID NO: 48 |
| 31 | Moderate | FLSTLSFVCPWCATN | SEQ ID NO: 49 |

K. HPV E6.52

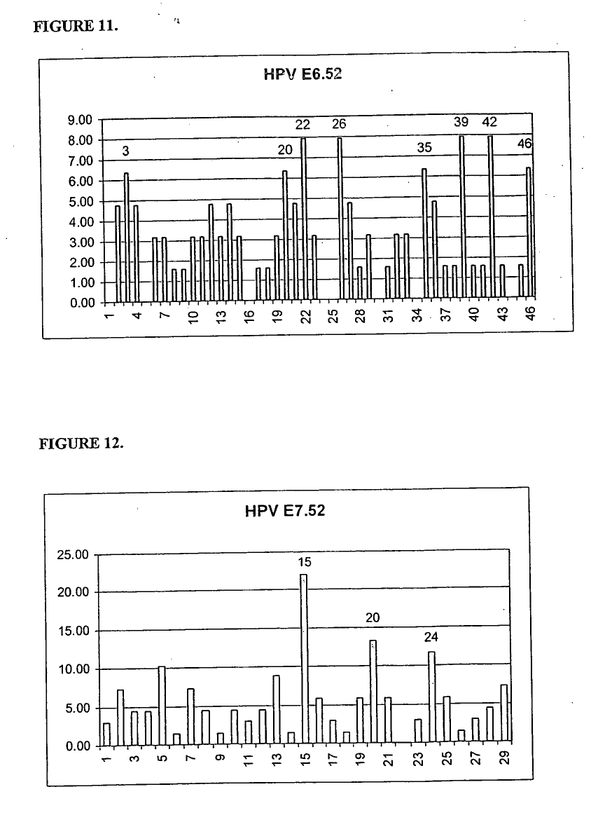

For this antigen, 63 donors were tested in the I-MUNE® assay to determine the epitope of interest for HPV E6.52. FIG. 11 provides a graph showing the responses to each epitope. Also as indicated in Table 10, there were 8 epitopes of interest identified in this antigen.

TABLE 10
HPV E6.52 Epitopes of Interest

| Peptide | Epitope | | |
|---|---|---|---|
| Number | Classification | Epitope Sequence | SEQ ID NO: |
| 3 | Minor | TRPRTLHELCEVLEE | SEQ ID NO: 50 |
| 20 | Minor | NPYGVCIMCLRFLSK | SEQ ID NO: 51 |
| 22 | Moderate | IMCLRFLSKISEYRH | SEQ ID NO: 52 |
| 26 | Moderate | YRHYQYSLYGKTLEE | SEQ ID NO: 53 |
| 35 | Minor | CIICQTPLCPEEKER | SEQ ID NO: 54 |
| 39 | Moderate | KERHVNANKRFHNIM | SEQ ID NO: 55 |
| 42 | Moderate | RFHNIMGRWTGRCSE | SEQ ID NO: 56 |
| 46 | Minor | GRCSECWRPRPVTQV | SEQ ID NO: 57 |

L. HPV E7.52

For this antigen, 68 donors were tested in the I-MUNE® assay to determine the epitopes of interest for HPV E7.52. FIG. 12 provides a graph showing the responses to each epitope. Also as indicated in Table 11, there were 3 epitopes of interest in this antigen.

TABLE 11
HPV E7.52 Epitopes of Interest

| Peptide | Epitope | | |
|---|---|---|---|
| Number | Classification | Epitope Sequence | SEQ ID NO: |
| 15 | Major | PDGQAEQATSNYYIV | SEQ ID NO: 58 |
| 20 | Minor | TYCHSCDSTLRLCIH | SEQ ID NO: 59 |
| 24 | Minor | CIHSTATDLRTLQQM | SEQ ID NO: 60 |

M. HPV E6.56

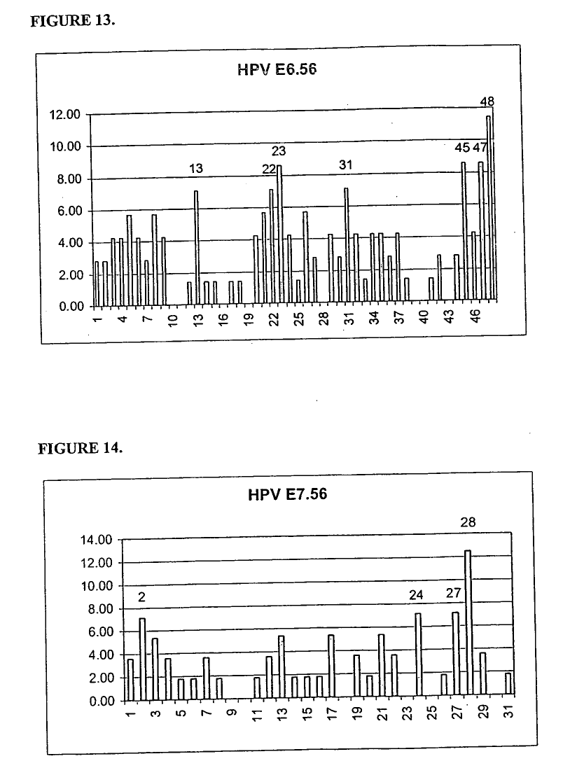

For this antigen, 70 donors were tested in the I-MUNE® assay to determine the epitopes of interest for HPV E6.56. FIG. 13 provides a graph showing the responses to each epitope. Also as indicated in Table 12, there were 7 epitopes of interest in this antigen.

TABLE 12
HPV E6.56 Epitopes of Interest

| Peptide | Epitope | | |
|---|---|---|---|
| Number | Classification | Epitope Sequence | SEQ ID NO: |
| 13 | Minor | KKELTRAEVYNFACT | SEQ ID NO: 61 |
| 22 | Minor | AVCRVCLLFYSKVRK | SEQ ID NO: 62 |
| 23 | Moderate | RVCLLFYSKVRKYRY | SEQ ID NO: 63 |
| 31 | Minor | LESITKKQLCDLLIR | SEQ ID NO: 64 |
| 45 | Moderate | HGWTGSCLGCWRQTS | SEQ ID NO: 65 |
| 47 | Moderate | CLGCWRQTSREPRES | SEQ ID NO: 66 |
| 48 | Major | GCWRQTSREPRESTV | SEQ ID NO: 67 |

N. HPV E7.56

For this antigen, 56 donors were tested in the 1-MUNE® assay to determine the epitopes of interest for HPV E7.56. FIG. 14 provides a graph showing the responses to each epitope. Also as indicated in Table 13, there were 4 epitopes of interest in this antigen.

TABLE 13
HPV E7.56

| Peptide | Epitope | | |
|---|---|---|---|
| Number | Classification | Epitope Sequence | SEQ ID NO: |
| 2 | Minor | KVPTLQDVVLELTPQ | SEQ ID NO: 68 |
| 24 | Minor | FVVQLDIQSTKEDLR | SEQ ID NO: 69 |
| 27 | Minor | TKEDLRVVQQLLMGA | SEQ ID NO: 70 |
| 28 | Major | DLRVVQQLLMGALTV | SEQ ID NO: 71 |

O. HPV E6.58

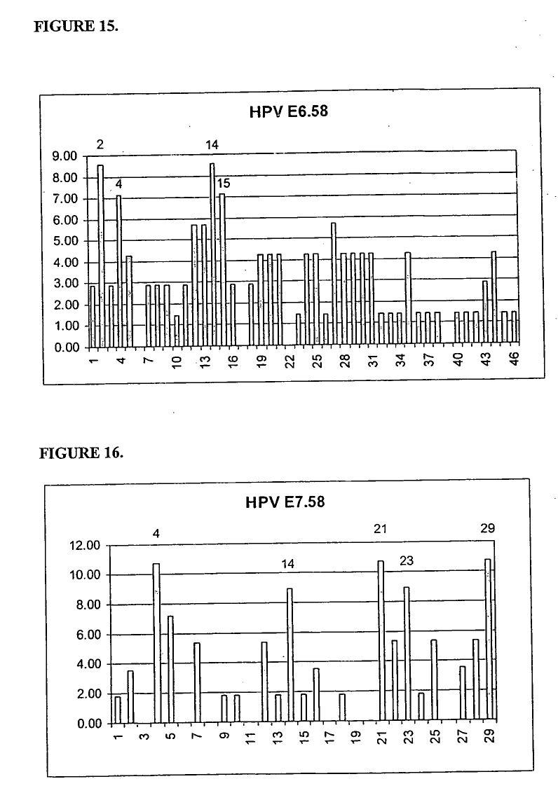

For this antigen, 70 donors were tested in the I-MUNE® assay to determine the epitopes of interest for HPV E6.58. FIG. 15 provides a graph showing the responses to each epitope. Also as indicated in Table 14, there were 4 epitopes of interest in this antigen.

TABLE 14
HPV E6.58 Epitopes of Interest

| Peptide | Epitope | | |
|---|---|---|---|
| Number | Classification | Epitope Sequence | SEQ ID NO: |
| 2 | Moderate | DAEEKPRTLHDLCQA | SEQ ID NO: 72 |
| 4 | Minor | RTLHDLCQALETSVH | SEQ ID NO: 73 |
| 14 | Moderate | SEVYDFVFADLRIVY | SEQ ID NO: 74 |
| 15 | Minor | YDFVFADLRIVYRDG | SEQ ID NO: 75 |

P. HPV E7.58

For this antigen, 56 donors were tested in the I-MUNE® assay to determine the epitopes of interest for HPV E7.58. FIG. 14 provides a graph showing the responses to each epitope. Also, as indicated in Table 15, there were 5 epitopes of interest in this antigen.

TABLE 15
HPV E7.58 Epitopes of Interest

| Peptide | Epitope | | |
|---|---|---|---|
| Number | Classification | Epitope Sequence | SEQ ID NO: |
| 4 | Moderate | EYILDLHPEPTDLFC | SEQ ID NO: 76 |
| 14 | Minor | DGPDGQAQPATANYY | SEQ ID NO: 77 |
| 21 | Moderate | TCGTTVRLCINSTTT | SEQ ID NO: 78 |
| 23 | Minor | RLCINSTTTDVRTLQ | SEQ ID NO: 79 |
| 29 | Moderate | LMGTCTIVCPSCAQQ | SEQ ID NO: 80 |

Q. HPV E2.16

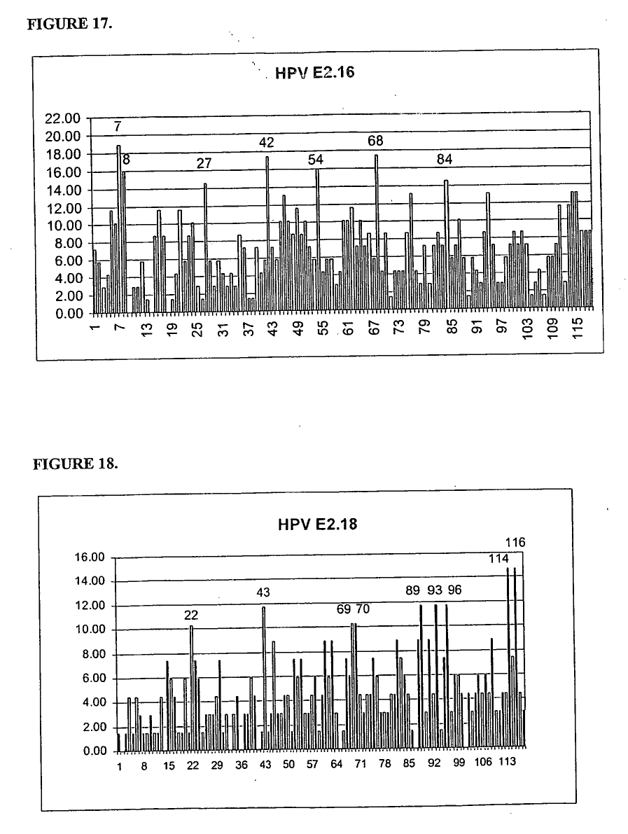

For this antigen, 69 donors were tested in the I-MUNE® assay to determine the epitope of interest for HPV E2.16. FIG. 17 provides a graph showing the responses to each epitope. Also, as indicated in Table 16, there were 7 epitopes of interest identified in this antigen.

TABLE 16
HPV E2.16 Epitopes of Interest

| Peptide | Epitope | | |
|---|---|---|---|
| Number | Classification | Epitope Sequence | SEQ ID NO: |
| 7 | Moderate | YENDSTDLRDHIDYW | SEQ ID NO: 81 |
| 8 | Moderate | DSTDLRDHIDYWKHM | SEQ ID NO: 82 |
| 27 | Minor | LETIYNSQYSNEKWT | SEQ ID NO: 83 |
| 42 | Moderate | DICNTMHYTNWTHIY | SEQ ID NO: 84 |
| 54 | Moderate | VHEGIRTYFVQFKDD | SEQ ID NO: 85 |
| 68 | Moderate | SNEVSSPEIIRQHLA | SEQ ID NO: 86 |
| 84 | Minor | PCHTTKLLHRDSVDS | SEQ ID NO: 87 |

R. HPV E2.18

For this antigen, 68 donors were tested in the I-MUNE® assay to determine the epitopes of interest for HPV E2.18. FIG. 18 provides a graph showing the responses to each epitope. Also as indicated in Table 17, there were 9 epitopes of interest identified in this antigen.

TABLE 17
HPV E2.18 Epitopes of Interest

| Peptide | Epitope | | |
|---|---|---|---|
| Number | Classification | Epitope Sequence | SEQ ID NO: |
| 22 | Minor | PAYNISKSKAHKAIE | SEQ ID NO: 88 |
| 43 | Moderate | GNKDNCMTYVAWDSV | SEQ ID NO: 89 |
| 69 | Minor | CSTSDDTVSATQLVK | SEQ ID NO: 90 |
| 70 | Minor | SDDTVSATQLVKQLQ | SEQ ID NO: 91 |
| 89 | Moderate | PLLGAATPTGNNKRR | SEQ ID NO: 92 |
| 93 | Moderate | KRRKLCSGNTTPIIH | SEQ ID NO: 93 |
| 96 | Moderate | TTPIIHLKGDRNSLK | SEQ ID NO: 94 |
| 114 | Major | ETQRTKFLNTVAIPD | SEQ ID NO: 95 |
| 116 | Major | FLNTVAIPDSVQILV | SEQ ID NO: 96 |

S. HPV E2.31

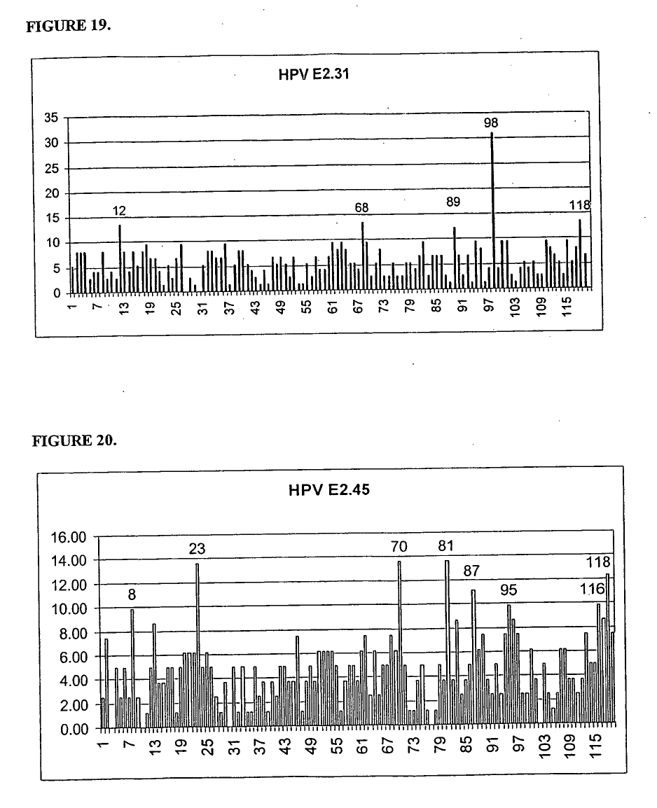

For this antigen, 74 donors were tested in the I-MUNE® assay to determine the epitopes of interest for HPV E2.31. FIG. 19 provides a graph showing the responses to each epitope. Also, as indicated in Table 18, there were 5 epitopes of interest identified in this antigen.

TABLE 18
LIPV E2.31 Epitopes of Interest

| Peptide | Epitope | | |
|---|---|---|---|
| Number | Classification | Epitope Sequence | SEQ ID NO: |
| 12 | Moderate | KHIRLECVLMYKARE | SEQ ID NO:97 |
| 68 | Moderate | SDEISFAGIVTKLPT | SEQ ID NO:98 |
| 89 | Minor | DSVDSVNCGVISAAA | SEQ ID NO:99 |
| 98 | Major | TTPIIHLKGDANILK | SEQ ID NO:100 |
| 118 | Moderate | DFLNTVKIPNTVSVS | SEQ ID NO:101 |

T. HPV E2.45

For this antigen, 74 donors were tested in the I-MUNE® assay to determine the epitopes of interest for HPV E2.45. FIG. 20 provides a graph showing the responses to each epitope. Also as indicated in Table 19, there were 8 epitopes of interest identified in this antigen.

TABLE 19
HPV E2.45 Epitopes of Interest

| Peptide | Epitope | | |
|---|---|---|---|
| Number | Classification | Epitope Sequence | SEQ ID NO: |
| 8 | Minor | LDHYENDSKDINSQI | SEQ ID NO:102 |
| 23 | Major | PINISKSKAHKAIEL | SEQ ID NO:103 |
| 70 | Major | STSDDTVSATQIVRQ | SEQ ID NO:104 |
| 81 | Major | PHIQTPATKRPRQCG | SEQ ID NO:105 |
| 87 | Moderate | QHHGRVNTHVHNPLL | SEQ ID NO:106 |
| 95 | Minor | RKVCSGNTTPIIHLK | SEQ ID NO:107 |
| 116 | Minor | RNTFLDVVTIPNSVQ | SEQ ID NO:108 |
| 118 | Moderate | VVTIPNSVQISVGYM | SEQ ID NO:109 |

We claim:

1. A method for the identification of CD4+ human papillomavirus epitopes.

2. A method for determining a T-cell epitope of a human papillomavirus strain, comprising the steps of:
(a) obtaining from a solution of dendritic cells and a solution of naïve CD4+ and/or CD8+ T-cells from a single human blood source;
(b) differentiating said dendritic cells, in said solution of dendritic cells, to produce a solution of differentiated dendritic cells;
(c) preparing a pepset of peptides from said human papillomavirus;
(d) combining said solution of differentiated dendritic cells and said naïve CD4+ and/or CD8+ T-cells with said pepset, wherein said pepset comprises said T-cell epitope; and
(e) measuring the proliferation of said T-cells in said step (d).

3. The method of claim 1, wherein said human papillomavirus (HPV) strain is selected from the group consisting of HPV16, HPV18, HPV31, HPV45, HPV52, HPV56, and HPV58.

4. The method of claim 2, wherein said protein comprises an amino acid sequence selected from the group consisting of SEQ ID NO:1 through SEQ ID NO:109.

5. The method of claim 2, wherein said pepset comprises a peptide having the sequence selected from the group consisting of SEQ ID NO:1 through SEQ ID NO:109.

6. A human papillomavirus CD4+ T-cell epitope identified using the method of claim 1 or 2.

7. A composition comprising the human papillomavirus CD4+ T-cell epitope of claim 6.

8. The method of claim 1, further comprising the step of modifying said human papillomavirus to produce a variant human papillomavirus, wherein said variant protein exhibits an altered immunogenic response as compared to said human papillomavirus.

9. A variant human papillomavirus produced according to the method of claim 8.

10. A composition comprising the variant protein of claim 9.
